(19) United States
(12) Patent Application Publication
(10) Pub. No.: US 2022/0211880 A1
(43) Pub. Date: Jul. 7, 2022

(54) IMAGING WITH LIPOSOME-BASED CONTRAST AGENTS BASED ON MODULATION OF MEMBRANE WATER PERMEABILITY

(71) Applicant: Massachusetts Institute of Technology, Cambridge, MA (US)

(72) Inventors: Alan Pradip Jasanoff, Cambridge, MA (US); Jacob Simon, Cambridge, MA (US)

(73) Assignee: Massachusetts Institute of Technology, Cambridge, MA (US)

(21) Appl. No.: 17/520,133

(22) Filed: Nov. 5, 2021

Related U.S. Application Data
(60) Provisional application No. 63/134,717, filed on Jan. 7, 2021.

(51) Int. Cl. A61K 49/18; A61K 47/24; A61K 47/28; A61K 49/10

(52) U.S. Cl. CPC A61K 49/1812; A61K 49/108; A61K 47/28; A61K 47/24

(57) ABSTRACT

The present disclosure provides imaging and contrast agents, and methods of using the agents. According to some embodiments of the disclosure, agents and methods for magnetic resonance imaging and related technologies are provided.

### RELATED APPLICATIONS

This application claims priority under 35 U.S.C. § 119(e) to U.S. Provisional Application No. 63/134,717, filed Jan. 7, 2021, and entitled “IMAGING WITH LIPOSOME-BASED CONTRAST AGENTS BASED ON MODULATION OF MEMBRANE WATER PERMEABILITY,” which is incorporated herein by reference in its entirety for all purposes.

### GOVERNMENT SPONSORSHIP

This invention was made with Government support under Grant No. R21 DA044748, R01 DA038642, and UF1 NS107712 awarded by the National Institutes of Health (NIH). The Government has certain rights in the invention.

### BACKGROUND

MRI is an attractive methodology for molecular imaging because of its almost unlimited depth penetration and relatively high spatiotemporal resolution. Molecular MRI probes have been developed for targets including small molecules, ions, enzymes, and light, but many of these agents exhibit comparatively poor sensitivity to their analytes. This is particularly true of paramagnetic contrast agents visualized via their effects on longitudinal relaxation time (T₁)-weighted MRI signals. T₁ agents induce favorable signal-brightening effects and are by far the most commonly used in the clinic, but they must usually be present at concentrations well into the micromolar range in order to produce substantial changes in image contrast. This poses a particular problem in sensing applications, including photodetection, where the amount of analyte is itself often limiting.

Most existing MRI sensors have been based on T₁ contrast mechanisms in which interaction of an analyte with a single sensing or binding moiety modulates inner-sphere magnetic interactions of a single paramagnetic ion with surrounding water molecules. This one-to-one actuation alters the strength of the agent's contrast effect, quantified by its longitudinal relaxivity (r₁), defined as the slope of relaxation rate (R₁=1/T₁) versus contrast agent concentration. Light-sensitive T₁ agents have followed this principle, wherein photoisomerization or photocleavage of a single chemical group alters the relaxation dynamics arising from an individual paramagnetic center. Such agents provide limited sensitivity to light and have not been demonstrated to enable photodetection in biological tissue.

Positron emission tomography (PET) and functional imaging by blood oxygenation level-dependent (BOLD) magnetic resonance imaging (MRI) allow noninvasive imaging of physiological variables in deep tissue, but both methods suffer from limitations. PET detects radioactive probes whose spatial distribution can be measured with high sensitivity, but the probes cannot be regulated, meaning that reversible detection of biological phenomena is generally impossible. Meanwhile, MRI-based functional imaging methods that use intrinsic contrast changes such as the BOLD effect offer only limited insight into the molecular or cellular bases of biological phenomena.

In addition, optical techniques for neuroimaging include brightfield and fluorescence microscopy, optogenetic stimulation and readouts, and optical coherence microscopy and tomography. These techniques are limited in scope to small, invasively exposed regions. Lack of whole-brain imaging capability and the potential interference with function of the invasive imaging limit their utility.

Moreover, known contrast agents for molecular MRI (e.g., synthetic molecules, proteins, nanoparticles) function to brighten or darken an MRI image by virtue of their magnetic properties in a manner that is dependent on a molecular phenomena of interest. However, these contrast agents are limited by the magnitude of signal changes generated and analyte sensitivity.

### SUMMARY

The present disclosure stems from the recognition that specific phenomena of interest can be linked to changes in MRI contrast, allowing for the effective imaging of mechanisms of biological function across molecular, cellular, and tissue-level scales. The agents, compositions, and methods disclosed herein function to relay signals of interest (e.g., of biological function) into MRI readouts, taking advantage of the essentially unlimited imaging depth and noninvasive nature of MRI.

By using paramagnetic liposomes as a contrast agent, whereby the effective brightness of a large, concentrated pool of encapsulated contrast agents are modulated at the liposomal membrane, the present disclosure provides dramatic signal amplification relative to existing imaging probes. For example, known T1 contrast agents require micromolar concentrations in order to be detected, whereas the paramagnetic liposomes of the present disclosure are capable of generating signal changes in excess of 50% at low nanomolar sensor concentrations. This enhanced sensitivity significantly expands the utility of MRI imaging techniques such that large contrast changes can be generated using sensor concentrations far below the concentration of analyte being measured, decreasing analyte buffering effects that confounds the interpretation of experimental results. In some embodiments, the low nanomolar sensor concentrations required also allows for the use of targeting domains, which use protein-protein interactions with endogenous or exogenously-expressed proteins that are localized near sites of interest, for example sites of analyte release, to enhance local sensor concentrations and further enhance analyte sensitivity.

To solve the limitations of responsive MRI probes that employ one-to-one actuation, the present disclosure provides a mechanism in which a relatively small number of analyte-sensing moieties regulate an abundance of paramagnetic centers simultaneously, resulting in one-to-many actuation and a corresponding amplification of responses.

In one aspect, provided are MRI imaging agents comprising: a liposome encapsulating an MRI contrast agent; and an analyte-recognition entity associated with the liposome that reversibly affects water permeability of the liposome in response to proximity of an analyte, or an electromagnetic radiation-affected entity associated with the liposome that reversibly affects water permeability of the liposome in response to electromagnetic radiation. In certain embodiments, the MRI imaging agents comprise a liposome encapsulating an MRI contrast agent; and an analyte-recognition entity associated with the liposome that reversibly affects water permeability of the liposome in response to proximity of an analyte.

In another aspect, provided are paramagnetic contrast agents comprising: a liposome; and an analyte-recognition entity associated with the liposome that reversibly affects water permeability of the liposome in response to proximity of an analyte, or an electromagnetic radiation-affected entity associated with the liposome that reversibly affects water permeability of the liposome in response to electromagnetic radiation. In certain embodiments, the paramagnetic contrast agents comprise a liposome; and an analyte-recognition entity associated with the liposome that reversibly affects water permeability of the liposome in response to proximity of an analyte.

In another aspect, provided are methods comprising: in a medium, exposing an analyte to an MRI contrast agent comprising a recognition entity for the analyte, wherein the concentration of the MRI contrast agent is less than 1 micromolar; and recording an MRI signal influenced by the contrast agent in the presence of the analyte that is at least 1% different from an MRI signal influenced by the contrast agent under essentially identical conditions but in the absence of the analyte. In certain embodiments, the analyte-recognition entity reversibly affects water permeability of the liposome in response to proximity of the analyte.

In another aspect, provided are methods of magnetic resonance imaging, the method comprising: in a medium, exposing an analyte or electromagnetic radiation to an MRI imaging agent or paramagnetic contrast agent of the present disclosure, wherein the concentration of the contrast agent is less than 1 micromolar; and recording an MRI signal induced by the contrast agent in the presence of the analyte or electromagnetic radiation that is at least 1% different from an MRI signal induced by the contrast agent under essentially identical conditions but in the absence of the analyte or electromagnetic radiation.

In another aspect, provided are kits comprising an MRI imaging agent or paramagnetic contrast agent and instructions for use.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures. In cases where the present specification and a document incorporated by reference include conflicting and/or inconsistent disclosure, the present specification shall control.

### BRIEF DESCRIPTION OF THE DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A to 1D depict analyte sensing with LisNRs. (FIG. 1A) Schematic demonstrating the operating principle of LisNRs. Inset shows hypothetical spatially-averaged signal. (FIG. 1B) Schematic demonstrating a competitive analyte sensing mechanism based on gramicidin A (gA) water-permeable pores. (FIG. 1C) Addition of biotinylated gA to paramagnetic liposomes (˜30 nM) increases R1 and MRI signal (inset) in a concentration-dependent manner. (FIG. 1D) Relative R1 change after addition of streptavidin (SA) to control liposomes and liposomes containing gA-biotin. For 3 nM liposome samples, the final concentration of gA-biotin and SA were 2 μM and 4 μM, respectively. For 30 nM liposome samples, the final concentrations of gA-biotin and SA were 20 μM and 33.3 μM, respectively.

FIGS. 2A to 2E depict liposomal nanoparticle reporters. (FIG. 2A) LisNRs incorporate high concentrations of liposome-encapsulated contrast agents such as gadoteridol, shown here. (FIG. 2B) Regulation of LisNR membrane permeability to water molecules underlies their performance as sensors. Lower membrane permeability suppresses water exchange rate, effective relaxivity (r_{1eff}), and T₁-weighted MRI signal (left), while higher permeability boosts water exchange, r_{1eff}, and MRI signal (right). (FIG. 2C) Model calculations indicate that substantial changes in effective relaxivity can be produced by modulating permeability over a physically plausible range. Results shown for three LisNR diameters. (FIG. 2D) Lipid composition influences membrane water permeability. Fully saturated lipids such as DPPC suppress permeability (top), while incorporation of unsaturated lipids like POPC (gray) promotes water exchange (bottom). (FIG. 2E) Liposomes encapsulating 220 mM gadoteridol and formulated with increasing percentages of POPC show systematically higher T₁w MRI contrast (top) and corresponding R₁ values (bottom).

FIGS. 3A to 3G depict construction and performance of Light LisNR in vitro. (FIG. 3A) Reversible photoisomerization of AzoPC is achieved using UV and blue light. In the blue light-activated state AzoPC adopts an extended conformation (top left) that emulates saturated lipids and reduces membrane water permeability (bottom left). In the UV-activated state, AzoPC exhibits a kinked structure (top right) that emulates unsaturated lipids and increases membrane permeability (bottom right). (FIG. 3B) Initial Light LisNRs exhibit light-dependent switching over repeated cycles of UV and blue illumination. The first, third, and fifth exposures are with UV light. The second, fourth, and sixth exposures are with blue light. (FIG. 3C) LisNRs formulated with different lipid compositions produce varying relaxation effects and sensitivity to temperature, as indicated by comparison of measurements at 22° C. and 38° C. From left to right, the seven compositions contain 85% DPPC, 10% AzoPC, and 5% DSPE-PEG; 65% DPPC, 20% POPC, 10% AzoPC, and 5% DSPE-PEG; 80% DPPC, 10% AzoPC, and 10% DPPG; 70% DPPC, 10% AzoPC, and 20% DPPG; 60% DPPC, 10% AzoPC, 10% DPPG, and 20% cholesterol; 40% DPPC, 10% AzoPC, 10% DPPG, and 40% cholesterol; 30% DPPC, 20% AzoPC, 10% DPPG, and 40% cholesterol. (FIG. 3D) LisNRs formulated with 30% DPPC, 20% AzoPC, 10% DPPG, and 40% cholesterol exhibited substantially enhanced efficacy compared with the initial variants in FIG. 3B. The first, third, and fifth exposures are with UV light. The second, fourth, and sixth exposures are with blue light. (FIG. 3E) Light scattering data indicating the mean Light LisNR diameter of 100 nm. (FIG. 3F) Light sensing in vitro with LisNRs. A solution (3 nM) of AzoPC-containing liposomes (20% AzoPC) were exposed to multiple cycles of UV (365 nm) and blue (460 nm) light which resulted in significant reversible changes in R1 and MRI signal (inset). For each cycle, the solution was exposed first to the UV light followed by the blue light. (FIG. 3G) Mean R1 of samples from FIG. 3F in cis (right) and trans (left) conformations.

FIGS. 4A to 4I show validation of Light LisNRs in rat brains. (FIG. 4A) Schematic of geometry for LisNR infusion into rat striatum and optical stimulation via an implanted fiber. (FIG. 4B) Mean image cross section indicates LisNR spreading over a region of roughly 2 mm full width at half height in rat brain. Scale bar=50% MRI signal difference. Inset shows an example injected slice with position of cross section for quantification indicated by dashed line. Scale bar=2 mm. (FIG. 4C) Signal difference observed following UV (top) or blue (bottom) illumination in an injected rat. Closed arrow indicates position of light guide, while open arrow denotes symmetrical site without fiber. (FIG. 4D) Mean light-responsive signal difference maps over a region corresponding to the dashed square in (FIG. 4C), averaged over three animals. (FIG. 4E) Time course of signal change exhibited near fiber tip before, during, and after UV illumination of Light LisNRs (n=3) or corresponding illumination of unresponsive control liposomes (gray, n=2). Black traces show unilluminated control data from Light LisNRs injected contralaterally to the fiber (n=3). Shading denotes SEM over animals. (FIG. 4F) Corresponding time courses obtained before, during, and after blue illumination. n values equivalent to FIG. 4E. (FIG. 4G) Signal differences observed from three animals under the conditions in (FIG. 4E) and (FIG. 4F). (FIG. 4H) MRI signal changes observed from two animals over repeated cycles of blue and UV illumination. Shading=SEM, black traces from contralateral control LisNR infusion sites. (FIG. 4I) Scatter plots comparing voxel level signal differences observed following illumination epochs labeled in (FIG. 4H).

FIGS. 5A to 5D show response mapping using LisNRs. (FIG. 5A) Map of blue light response amplitude in a gel phantom formulated with Light LisNRs (left). Location of the fiber indicated by label. Traces at right indicate time course of image signal change during illumination at spatial positions denoted by dashed lines. Scale bars=2 s (horizontal) and 20% (vertical). (FIG. 5B) Photon flux modeled using a beam spreading model used to approximate illumination profiles in phantom experiments. Dashed outer box corresponds to the region denoted equivalently in (FIG. 5A). Dashed inner box corresponds to the slice thickness in (FIG. 5A), over which light responses are integrated to model the observed signal changes. (FIG. 5C) Comparison of light response rates (k) computed from data (left) or beam spreading models (right) for UV illumination (top) and blue illumination (bottom). (FIG. 5D) Map of blue light response in a single rat brain, with close-up (bottom) corresponding to dashed box in the full coronal slice (top). Traces at right denote signal time courses during illumination at positions indicated by dashed lines. Scale bars=2 s (horizontal) and 5% (vertical). (FIG. 5E) Maps of response rates in the vicinity fiber tips (vertical line), showing variability among three animals (columns) and differences between UV (top) and blue (bottom) illumination responses. (FIG. 5F) Mean response rates to illumination among six animals under UV and blue illumination conditions. (FIG. 5G) Photon flux modeled as a combination of beam spreading (top) and photon diffusion (bottom) from fibers (vertical lines). Dashed boxes denote regions corresponding to fields of view in the image closeups in (FIG. 5E), both in-plane (outer white) and orthogonal to image slices (inner). (FIG. 5H) Comparison of k values computed from mean data (left) and optimized hybrid models (right) showing approximate agreement.

### DETAILED DESCRIPTION OF CERTAIN EMBODIMENTS

### Imaging Agents

Disclosed herein are Liposomal Nanoparticle Reporters (LisNRs), a novel class of contrast agent for functional imaging in vivo of tissue and organs (e.g., the brain). In certain embodiments, a functionally relevant molecular or cellular phenomenon is detected through reversible modulation of the water permeability of the lipid bilayer of a paramagnetic liposomal contrast agent that permits detection of these phenomenon via MRI contrast changes. In certain embodiments, the agents comprise a concentrated pool of conventional T1-weighted MRI contrast agents encapsulated within a synthetic liposome. T1-weighted MRI contrast agents primarily alter MRI signal through direct interactions between paramagnetic metal ions and water protons. MRI sensors based on this contrast mechanism operate by introducing changes in water access to a paramagnetic ion upon analyte binding. With LisNRs, such changes in water access are accomplished by modulating the water permeability of liposomal bilayers that enclose the concentrated pool of encapsulated T1-weighted contrast agents, thereby modulating MRI contrast of many contrast agents at once. In certain embodiments, the liposomal bilayer permeability is dependent on a molecular or cellular phenomenon of interest, permitting the phenomenon to be detected by means of an MRI signal change. In certain embodiments, the molecular or cellular phenomenon detected may be the concentration of a physiological analyte or a biological activity, such as gene expression or secretion. The liposomal contrast agent described relays the biological signal into a change in MRI signal.

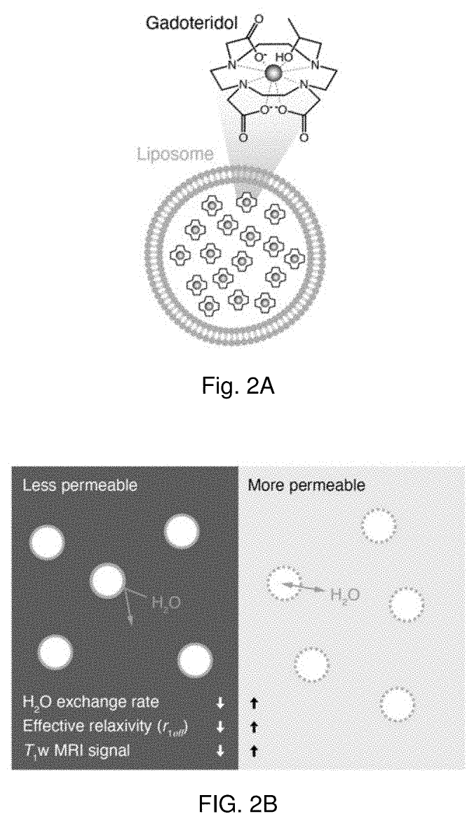

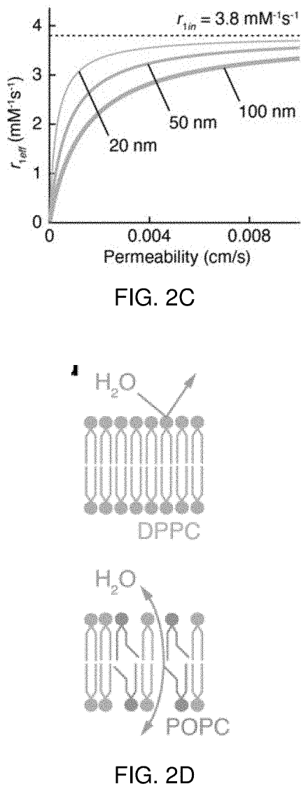

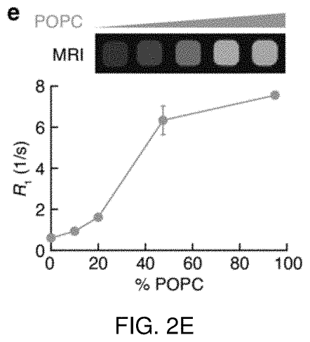

For example, encapsulation of paramagnetic MRI contrast agents such as gadoteridol in liposomes (FIG. 2A) is known to decrease their effective r₁, due to reduced hydrodynamic exchange between the paramagnetic metal centers and the surrounding bulk solvent. Liposomes with lower solvent permeability experience reduced water exchange and lower effective r₁ per metal ion (r_{1eff}), and produce lower T₁-weighted MRI signal than liposomes with higher permeability (FIG. 2B). Modulation of membrane permeability over a physically plausible range from 0 to 0.01 cm/s is expected to yield r_{1eff} values from 0 to nearly 3.8 mM⁻¹s⁻¹, the r₁ of unencapsulated gadoteridol at 7 T and room temperature (FIG. 2C).

Liposomal membranes that consist of closely packed, saturated lipids tend to block water exchange and reduce relaxivity, whereas unsaturated, fluidizing lipids facilitate water exchange and higher T₁ relaxation effects (FIG. 2D). Dramatic differences in MRI contrast thus arise from 100 nm-diameter liposomes formulated with varying mixes of the fluidizing lipid 1-palmitoyl-2-oleoyl-sn-glycero-3-phosphocholine (POPC) and the non-fluidizing 1,2-dipalmitoyl-sn-glycero-3-phosphocholine (DPPC) and containing 220 mM gadoteridol (FIG. 2E). Such liposomes enclose tens of thousands of contrast agent molecules, thus a concerted mechanism for reversibly regulating their permeability provides a means for highly amplified analyte sensing in molecular MRI.

Accordingly, disclosed herein are imaging and/or contrast agents useful in magnetic resonance imaging.

In one aspect, disclosed is an MRI imaging agent comprising: a liposome encapsulating an MRI contrast agent; and an analyte-recognition entity associated with the liposome that reversibly affects water permeability of the liposome in response to proximity of an analyte, or an electromagnetic radiation-affected entity associated with the liposome that reversibly affects water permeability of the liposome in response to electromagnetic radiation.

In certain embodiments, the MRI imaging agent comprises a liposome encapsulating an MRI contrast agent; and an analyte-recognition entity associated with the liposome that reversibly affects water permeability of the liposome in response to proximity of an analyte.

In certain embodiments, the MRI imaging agent comprises a liposome encapsulating an MRI contrast agent; and an electromagnetic radiation-affected entity associated with the liposome that reversibly affects water permeability of the liposome in response to electromagnetic radiation.

In another aspect, disclosed is a paramagnetic contrast agent comprising: a liposome; and an analyte-recognition entity associated with the liposome that reversibly affects water permeability of the liposome in response to proximity of an analyte, or an electromagnetic radiation-affected entity associated with the liposome that reversibly affects water permeability of the liposome in response to electromagnetic radiation.

In certain embodiments, the paramagnetic contrast agent comprises a liposome; and an analyte-recognition entity associated with the liposome that reversibly affects water permeability of the liposome in response to proximity of an analyte.

In certain embodiments, the paramagnetic contrast agent comprises a liposome; and an electromagnetic radiation-affected entity associated with the liposome that reversibly affects water permeability of the liposome in response to electromagnetic radiation.

### Liposome

In certain embodiments, the liposome is a synthetic liposome. In certain embodiments, the liposome comprises a phospholipid, cholesterol, or a combination thereof. In certain embodiments, the liposome comprises a phosphatidylglycerol, a phosphatidylethanolamine, a phosphatidylcholine, a phosphatidylserine, or cholesterol, or a combination thereof. In certain embodiments, the liposome comprises a phosphatidylglycerol, a phosphatidylcholine, or cholesterol, or a combination thereof.

In certain embodiments, the liposome comprises a phospholipid comprising a photoisomerizable moiety. In certain embodiments, the photoisomerizable moiety is an azobenzene. In certain embodiments, the photoisomerizable moiety is 1-stearoyl-2-(4-(n-butyl)phenylazo-4′-phenylbutyroyl)phosphocholine (AzoPC).

In certain embodiments, the phosphatidylcholine is 1,2-Dipalmitoyl-sn-Glycero-3-Phosphocholine (DPPC), 1-palmitoyl-2-oleoyl-sn-glycero-3-phosphocholine (POPC), or 1-stearoyl-2-(4-(n-butyl)phenylazo-4′-phenylbutyroyl)phosphocholine (AzoPC), or a combination thereof. In certain embodiments, the phosphatidylcholine is 1,2-Dipalmitoyl-sn-Glycero-3-Phosphocholine (DPPC), or 1-stearoyl-2-(4-(n-butyl)phenylazo-4′-phenylbutyroyl)phosphocholine (AzoPC), or a combination thereof. In certain embodiments, the phosphatidylcholine is 1,2-Dipalmitoyl-sn-Glycero-3-Phosphocholine (DPPC) and 1-stearoyl-2-(4-(n-butyl)phenylazo-4′-phenylbutyroyl)phosphocholine (AzoPC). In certain embodiments, the phosphatidylcholine is 1,2-Dipalmitoyl-sn-Glycero-3-Phosphocholine (DPPC). In certain embodiments, the phosphatidylglycerol is 1,2-Dipalmitoyl-sn-Glycero-3-Phosphoglycerol (DPPG). In certain embodiments, the phosphatidylethanolamine is Polyethylene Glycol-Modified 1,2-Distearoyl-sn-Glycero-3-Phosphoethanolamine (DSPE-PEG).

In certain embodiments, the liposome comprises, by weight, about 20% to about 99%, about 20% to about 95%, about 20% to about 90%, about 20% to about 80%, about 20% to about 70%, about 20% to about 60%, about 20% to about 50%, about 30% to about 95%, about 40% to about 95%, about 50% to about 95%, about 30% to about 70%, about 30% to about 60%, about 30% to about 50%, about 40% to about 95%, about 40% to about 80%, about 40% to about 70%, about 40% to about 60%, about 40% to about 50%, or about 50% phosphatidylcholine. In certain embodiments, the liposome comprises, by mol %, about 20% to about 99%, about 20% to about 95%, about 20% to about 90%, about 20% to about 80%, about 20% to about 70%, about 20% to about 60%, about 20% to about 50%, about 30% to about 95%, about 40% to about 95%, about 50% to about 95%, about 30% to about 70%, about 30% to about 60%, about 30% to about 50%, about 40% to about 95%, about 40% to about 80%, about 40% to about 70%, about 40% to about 60%, about 40% to about 50%, or about 50% phosphatidyicholine.

In certain embodiments, the liposome comprises, by weight, about 5% to about 30%, about 5% to about 20%, about 5% to about 10%, about 10% to about 30%, about 20% to about 30%, about 10% to about 20%, about 5% to about 15%, about 15% to about 25%, about 10%, or about 20% phosphatidylglycerol. In certain embodiments, the liposome comprises, by mol %, about 5% to about 30%, about 5% to about 20%, about 5% to about 10%, about 10% to about 30%, about 20% to about 30%, about 10% to about 20%, about 5% to about 15%, about 15% to about 25%, about 10%, or about 20% phosphatidylglycerol.

In certain embodiments, the liposome comprises, by weight, about 1% to about 30%, about 1% to about 20%, about 1% to about 10%, about 1% to about 5%, about 5% to about 30%, about 5% to about 20%, about 5% to about 15%, about 5% to about 10%, about 5%, or about 10% phosphatidylethanolamine. In certain embodiments, the liposome comprises, by mol %, about 1% to about 30%, about 1% to about 20%, about 1% to about 10%, about 1% to about 5%, about 5% to about 30%, about 5% to about 20%, about 5% to about 15%, about 5% to about 10%, about 5%, or about 10% phosphatidylethanolamine.

In certain embodiments, the liposome comprises, by weight, about 10% to about 60%, about 10% to about 50%, about 10% to about 30%, about 10% to about 20%, about 20% to about 60%, about 20% to about 50%, about 20% to about 40%, about 20% to about 30%, about 30% to about 40%, about 30% to about 50%, about 40%, or about 20% cholesterol. In certain embodiments, the liposome comprises, by mol %, about 10% to about 60%, about 10% to about 50%, about 10% to about 30%, about 10% to about 20%, about 20% to about 60%, about 20% to about 50%, about 20% to about 40%, about 20% to about 30%, about 30% to about 40%, about 30% to about 50%, about 40%, or about 20% cholesterol.

In certain embodiments, the liposome comprises about 85% DPPC, about 10% AzoPC, and about 5% DSPE-PEG. In certain embodiments, the liposome comprises, by weight, about 85% DPPC, about 10% AzoPC, and about 5% DSPE-PEG. In certain embodiments, the liposome comprises, by mol %, about 85% DPPC, about 10% AzoPC, and about 5% DSPE-PEG.

In certain embodiments, the liposome comprises about 65% DPPC, 20% POPC, about 10% AzoPC, and about 5% DSPE-PEG. In certain embodiments, the liposome comprises, by weight, about 65% DPPC, 20% POPC, about 10% AzoPC, and about 5% DSPE-PEG. In certain embodiments, the liposome comprises, by mol %, about 65% DPPC, 20% POPC, about 10% AzoPC, and about 5% DSPE-PEG.

In certain embodiments, the liposome comprises about 80% DPPC, about 10% AzoPC, and about 10% DPPG. In certain embodiments, the liposome comprises, by weight, about 80% DPPC, about 10% AzoPC, and about 10% DPPG. In certain embodiments, the liposome comprises, by mol %, about 80% DPPC, about 10% AzoPC, and about 10% DPPG.

In certain embodiments, the liposome comprises about 70% DPPC, about 10% AzoPC, and about 20% DPPG. In certain embodiments, the liposome comprises, by weight, about 70% DPPC, about 10% AzoPC, and about 20% DPPG. In certain embodiments, the liposome comprises, by mol %, about 70% DPPC, about 10% AzoPC, and about 20% DPPG.

In certain embodiments, the liposome comprises about 60% DPPC, about 10% AzoPC, about 10% DPPG, and about 20% cholesterol. In certain embodiments, the liposome comprises, by weight, about 60% DPPC, about 10% AzoPC, about 10% DPPG, and about 20% cholesterol. In certain embodiments, the liposome comprises, by mol %, about 60% DPPC, about 10% AzoPC, about 10% DPPG, and about 20% cholesterol.

In certain embodiments, the liposome comprises about 40% DPPC, about 10% AzoPC, about 10% DPPG, and about 40% cholesterol. In certain embodiments, the liposome comprises, by weight, about 40% DPPC, about 10% AzoPC, about 10% DPPG, and about 40% cholesterol. In certain embodiments, the liposome comprises, by mol %, about 40% DPPC, about 10% AzoPC, about 10% DPPG, and about 40% cholesterol.

In certain embodiments, the liposome comprises about 30% DPPC, about 20% AzoPC, about 10% DPPG, and about 40% cholesterol. In certain embodiments, the liposome comprises, by weight, about 30% DPPC, about 20% AzoPC, about 10% DPPG, and about 40% cholesterol. In certain embodiments, the liposome comprises, by mol %, about 30% DPPC, about 20% AzoPC, about 10% DPPG, and about 40% cholesterol.

In certain embodiments, the liposome comprises a molecular entity bound to a surface of the liposome. In certain embodiments, the molecular entity bound to the surface is covalently bound to one or more phospholipid comprising the liposome. In certain embodiments, the molecular entity bound to the surface is a targeting moiety. In certain embodiments, the targeting moiety is a polymer, a small molecule, a protein, a peptide, a nucleotide, a polynucleotide, a nucleic acid, or an antibody. In certain embodiments, the targeting moiety is a small molecule. In certain embodiments, the targeting moiety is a protein. In certain embodiments, the targeting moiety is a peptide. In certain embodiments, the targeting moiety is a nucleotide. In certain embodiments, the targeting moiety is a polynucleotide. In certain embodiments, the targeting moiety is a nucleic acid. In certain embodiments, the targeting moiety is an antibody. In certain embodiments, the targeting moiety is a polymer. In certain embodiments, the targeting moiety is a polyethylene glycol chain. In certain embodiments, the polyethylene glycol chain can prolong the circulation time, improve bioavailability, reduce undesirable side effects, and/or target specific cells, tissues, or intracellular localization.

In certain embodiments, the liposome does not degrade at a temperature of 40° C., 41° C., 42° C., 43° C., 44° C., or 45° C. In certain embodiments, less than 50% of the liposome degrades at a temperature of 40° C., 41° C., 42° C., 43° C., 44° C., or 45° C. In certain embodiments, less than 40% of the liposome degrades at a temperature of 40° C., 41° C., 42° C., 43° C., 44° C., or 45° C. In certain embodiments, less than 30% of the liposome degrades at a temperature of 40° C., 41° C., 42° C., 43° C., 44° C., or 45° C. In certain embodiments, less than 20% of the liposome degrades at a temperature of 40° C., 41° C., 42° C., 43° C., 44° C., or 45° C. In certain embodiments, less than 10% of the liposome degrades at a temperature of 40° C., 41° C., 42° C., 43° C., 44° C., or 45° C. In certain embodiments, less than 5% of the liposome degrades at a temperature of 40° C., 41° C., 42° C., 43° C., 44° C., or 45° C. In certain embodiments, less than 1% of the liposome degrades at a temperature of 40° C., 41° C., 42° C., 43° C., 44° C., or 45° C.

In certain embodiments, the liposome has a diameter of about 1 nm to about 500 nm, about 1 nm to about 400 nm, about 1 nm to about 300 nm, about 1 nm to about 200 nm, about 1 nm to about 100 nm, about 1 nm to about 90 nm, about 1 nm to about 80 nm, about 1 nm to about 70 nm, about 1 nm to about 60 nm, about 1 nm to about 50 nm, about 1 nm to about 40 nm, about 1 nm to about 30 nm, about 1 nm to about 20 nm, about 1 nm to about 10 nm, or about 1 nm to about 5 nm. In certain embodiments, the liposome has a diameter of at least 1 nm, at least 5 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, at least 400 nm, or at least 500 nm. In certain embodiments, the liposome has a diameter of equal to or less than 1 nm, equal to or less than 5 nm, equal to or less than 10 nm, equal to or less than 20 nm, equal to or less than 30 nm, equal to or less than 40 nm, equal to or less than 50 nm, equal to or less than 60 nm, equal to or less than 70 nm, equal to or less than 80 nm, equal to or less than 90 nm, equal to or less than 100 nm, equal to or less than 200 nm, equal to or less than 300 nm, equal to or less than 400 nm, or equal to or less than 500 nm.

In certain embodiments, the liposome comprises a water-permeable molecular channel spanning a lipid bilayer of the liposome. In certain embodiments, the water-permeable molecular channel contains or binds the analyte recognition entity. In certain embodiments, the water-permeable molecular channel contains the electromagnetic radiation-affected entity. In certain embodiments, the water-permeable molecular channel is or comprises an organic chemical structure. In certain embodiments, the chemical structure of the water-permeable molecular channel is different from the chemical structure of the remainder of the liposome. In certain embodiments, the water-permeable molecular channel is or comprises a transmembrane protein, a polypeptide, a phospholipid, a fatty acid chain, or a synthetic organic compound. In certain embodiments, the water-permeable molecular channel is or comprises a transmembrane protein. In certain embodiments, the water-permeable molecular channel is or comprises an aquaporin. In certain embodiments, the water-permeable molecular channel is or comprises a polypeptide. In certain embodiments, the water-permeable molecular channel is or comprises a phospholipid. In certain embodiments, the water-permeable molecular channel is or comprises a fatty acid chain. In certain embodiments, the water-permeable molecular channel is or comprises a synthetic organic compound. In certain embodiments, the water-permeable molecular channel comprises a phospholipid having a modified fatty acid chain. In certain embodiments, the modified fatty acid chain comprises a photoisomerizable moiety. In certain embodiments, the modified fatty acid chain comprises an azobenzene moiety. In certain embodiments, the water-permeable channel comprises 1-stearoyl-2-(4-(n-butyl)phenylazo-4′-phenylbutyroyl)phosphocholine (AzoPC).

In certain embodiments, the water-permeable molecular channel is impermeable to organic compounds. In certain embodiments, the water-permeable molecular channel has greater water permeability than organic compound permeability. In certain embodiments, the water-permeable molecular channel has water permeability that is at least 1.1, 1.2, 1.3, 1.4, 1.5, 1.6, 1.7, 1.8, 1.9, 2, 3, 4, 5, 10, 20, 30, 40, 50, 100, 200, 300, 400, 500, 1000, or 10000 times more permeable than its organic compound permeability. In certain embodiments, the water-permeable molecular channel has greater organic compound permeability than water permeability.

### Contrast Agent

In certain embodiments, the liposome is a contrast agent. In certain embodiments, the liposome comprises a contrast agent. In certain embodiments, the liposome encapsulates a contrast agent. In certain embodiments, the contrast agent is any contrast agent suitable for obtaining a magnetic resonance spectrum. In certain embodiments, the contrast agent is any contrast agent suitable for obtaining a magnetic resonance image. In certain embodiments, the contrast agent is paramagnetic. In certain embodiments, the contrast agent comprises a metal. In certain embodiments, the contrast agent comprises a metal ion. In certain embodiments, the contrast agent comprises particles (e.g., nanoparticles, microparticles). In certain embodiments, the contrast agent comprises iron, gadolinium, or manganese. In certain embodiments, the contrast agent comprises iron (III), gadolinium (III), or manganese (II). In certain embodiments, the contrast agent comprises gadolinium (III), superparamagnetic iron oxide, superparamagnetic iron-platinum particles, or manganese (II). In certain embodiments, the contrast agent comprises gadolinium (III). In certain embodiments, the contrast agent is or comprises gadoterate, gadobutrol, gadoteridol, gadopentetate, gadobenate, gadopentetic acid dimeglumine, gadoxentate, gadoxetic acid, gadodiamide, gadoversetamide, gadofosveset, gadocoletic acid, gadomelitol, or gadomer 17. In certain embodiments, the contrast agent is or comprises gadoteridol.

Agents that Modulate Liposomal Water Permeability Using Water-Permeable Channels

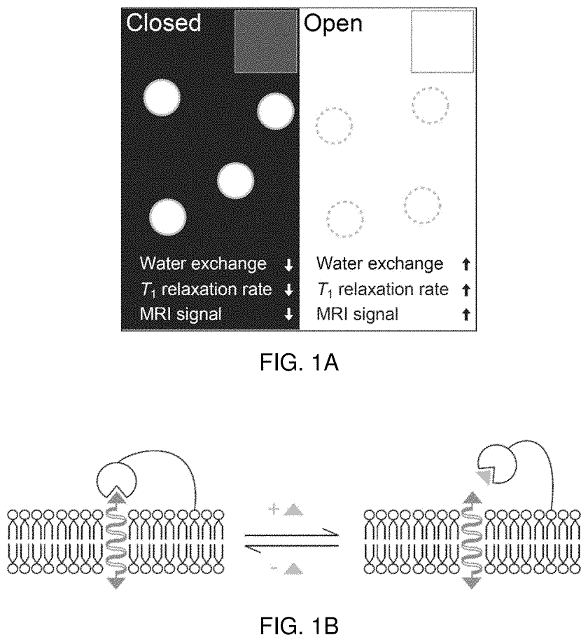

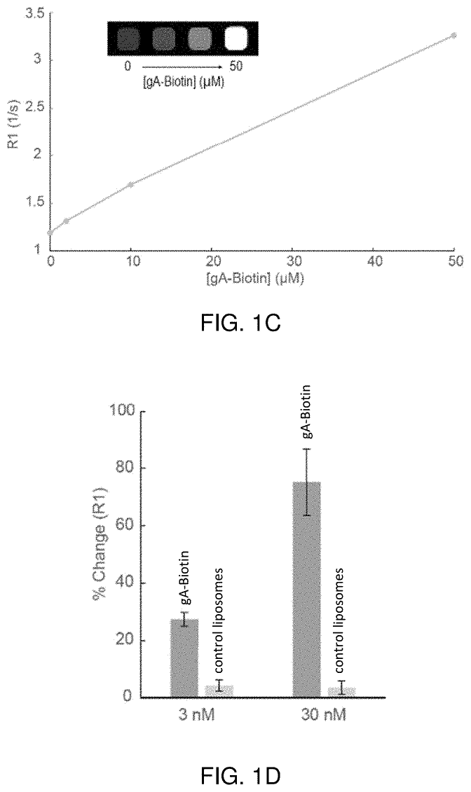

In one embodiment of the present disclosure, water permeability of the water-permeable molecular channel is regulated by response to phenomena of interest. A variety of naturally occurring peptide- and small molecule-based pores are capable of spontaneously forming transmembrane channels. As one example, gramicidin A spontaneously incorporates and form water-permeable pores in paramagnetic liposomes. Incorporation of gramicidin A channels does not cause large scale liposome disruption or leakage of encapsulated MRI contrast agent. In certain embodiments, a tethered neurotransmitter analog is conjugated to the channel and a corresponding neurotransmitter-binding protein is conjugated to the surface of the liposome, such that the protein can bind to the peptide resulting in decreased peptide activity and low liposomal bilayer water permeability. When sufficient levels of the neurotransmitter are present in solution, they compete with the modified peptide for the protein neurotransmitter-binding site, thus increasing the amount of unbound peptide and increasing the liposomal bilayer water permeability resulting in an increase in T1-weighted MRI signal (e.g., FIG. 1).

Accordingly, in certain embodiments, the liposome comprises a water-permeable channel that binds the analyte recognition entity. In certain embodiments, the water-permeable molecular channel is a polypeptide. In certain embodiments, the water-permeable molecular channel is gramicidin A. In certain embodiments, the liposome comprises a tethered moiety that binds the analyte recognition entity. In certain embodiments, the tethered moiety is conjugated to the water-permeable molecular channel. In certain embodiments, the tethered moiety is a synthetic compound or an endogenous compound. In certain embodiments, the tethered moiety is an analog of an endogenous compound. In certain embodiments, the tethered moiety is a neurotransmitter, a neurotransmitter analog, a sugar (e.g., glucose), a modified sugar, or a tumor-associated analyte. In certain embodiments, the tethered moiety is a neurotransmitter or neurotransmitter analog. In certain embodiments, the tethered moiety is dopamine or a dopamine analog. In certain embodiments, the tethered moiety is dopamine. In certain embodiments, the tethered moiety is a dopamine analog. In certain embodiments, the dopamine or dopamine analog binds the analyte-recognition entity.

In certain embodiments, the analyte-recognition entity is bound to the surface of the liposome. In certain embodiments, the analyte-recognition entity is a protein receptor. In certain embodiments, the analyte-recognition entity is a protein receptor and is bound to the surface of the liposome. In certain embodiments, the analyte-recognition entity binds the tethered moiety such that the water permeable molecular channel is less permeable to water when the analyte recognition entity is bound to the tethered moiety than when the analyte recognition is not bound to the tethered moiety.

Agents that Modulate Liposomal Membrane Fluidity

In another embodiment of the present disclosure, membrane water permeability of the liposome may be altered by modulating bilayer fluidity. In certain embodiments, modulating bilayer fluidity can be accomplished through induced conformational changes in lipid fatty acid chains that affect lipid-lipid interactions.

For example, a light-sensitive agent can be constructed with a phospholipid comprising a photoisomerizable moiety (e.g., AzoPC). One of the fatty acid chains of AzoPC contains a photoisomerizable azobenzene moiety. Irradiation with UV light favors the bulkier cis conformation increasing membrane fluidity and water permeability while thermal relaxation or irradiation with blue light favors the trans conformation decreasing membrane fluidity and water permeability.

Accordingly, in certain embodiments, the agent relies on electromagnetic radiation to induce a conformational change in a molecular entity that subsequently affects the bilayer fluidity (e.g., water permeability) of the liposomal membrane.

In certain embodiments, the water-permeable molecular channel contains the analyte-recognition entity. In such embodiments, the analyte-recognition entity is an electromagnetic radiation-affected entity, wherein the analyte-recognition entity is affected by electromagnetic radiation to switch between a first conformation providing a first level of water permeability across the lipid bilayer of the liposome, and a second conformation providing a second level of water permeability across the lipid bilayer. In certain embodiments, the analyte is a molecule that induces light emission of a light-emitting enzyme (e.g., luciferase) when the analyte is in proximity to the light-emitting enzyme. Thus, in certain embodiments, this agent can be used in combination with virally expressed photon-emitting luciferase enzymes and luciferase-based molecular sensors to transduce luciferase activity into MRI contrast.

In certain embodiments, the electromagnetic radiation-affected entity is not an analyte-recognition entity. In certain embodiments, the water-permeable molecular channel contains the electromagnetic radiation-affected entity. In certain embodiments, the electromagnetic radiation-affected entity is affected by electromagnetic radiation to switch between a first conformation providing a first level of water permeability across the lipid bilayer of the liposome, and a second conformation providing a second level of water permeability across the lipid bilayer.

In certain embodiments, the electromagnetic radiation is light. In certain embodiments, the electromagnetic radiation is light having a wavelength of about 10 nm to about 800 nm, about 10 nm to about 700 nm, about 10 nm to about 600 nm, about 10 nm to about 500 nm, about 10 nm to about 400 nm, about 10 nm to about 390 nm, about 10 nm to about 380 nm, about 10 nm to about 370 nm, about 100 nm to about 400 nm, about 100 nm to about 390 nm, about 100 nm to about 380 nm, about 100 nm to about 370 nm, about 300 nm to about 800 nm, about 300 nm to about 700 nm, about 300 nm to about 600 nm, about 300 nm to about 500 nm, about 300 nm to about 400 nm, about 300 nm to about 390 nm, about 300 nm to about 380 nm, about 300 nm to about 370 nm, about 330 nm to about 380 nm, about 350 nm to about 380 nm, about 350 nm to about 370 nm, about 360 nm to about 380 nm, about 360 nm to about 370 nm, about 365 nm to about 375 nm, about 365 nm to about 370 nm, about 365 nm, or about 370 nm.

### Methods

Also disclosed herein are methods for measuring and visualizing molecular concentrations or activities (“molecular imaging”), or aspects of cellular function (“cellular imaging”), in live animals or humans based on relaying these signals of interest into changes of MRI contrast through reversible, analyte-dependent changes in lipid bilayer water permeability of paramagnetic liposomal probes. In certain embodiments, the methods employ the imaging and/or contrast agents described herein. In certain embodiments, the methods are useful for the detection of physiologically relevant molecular species. In certain embodiments, the methods are useful for the visualization of spatial and/or temporal patterns of gene expression. In certain embodiments, the methods are useful for scientific investigation of physiological structures and phenomena. In certain embodiments, the methods are useful for the testing of substances for pharmacological or pharmacokinetic properties. In certain embodiments, the methods are useful clinical diagnostic imaging.

In one aspect, disclosed is a method comprising: in a medium, exposing an analyte to an MRI contrast agent comprising a recognition entity for the analyte, wherein the concentration of the MRI contrast agent is less than 1 micromolar; and recording an MRI signal influenced by the contrast agent in the presence of the analyte that is at least 1% different from an MRI signal influenced by the contrast agent under essentially identical conditions but in the absence of the analyte. In certain embodiments, the analyte-recognition entity reversibly affects water permeability of the liposome in response to proximity of the analyte.

In another aspect, disclosed is a method of magnetic resonance imaging, the method comprising: in a medium, exposing an analyte or electromagnetic radiation to an MRI imaging agent or paramagnetic contrast agent of the present disclosure, wherein the concentration of the contrast agent is less than 1 micromolar; and recording an MRI signal induced by the contrast agent in the presence of the analyte or electromagnetic radiation that is at least 1% different from an MRI signal induced by the contrast agent under essentially identical conditions but in the absence of the analyte or electromagnetic radiation.

In certain embodiments, the method comprises exposing an analyte or electromagnetic radiation to an MRI imaging agent of the present disclosure. In certain embodiments, the method comprises exposing an analyte to an MRI imaging agent of the present disclosure. In certain embodiments, the method comprises exposing electromagnetic radiation to an MRI imaging agent of the present disclosure.

In certain embodiments, the electromagnetic radiation is light. In certain embodiments, the electromagnetic radiation is light having a wavelength of about 10 nm to about 800 nm, about 10 nm to about 700 nm, about 10 nm to about 600 nm, about 10 nm to about 500 nm, about 10 nm to about 400 nm, about 10 nm to about 390 nm, about 10 nm to about 380 nm, about 10 nm to about 370 nm, about 100 nm to about 400 nm, about 100 nm to about 390 nm, about 100 nm to about 380 nm, about 100 nm to about 370 nm, about 300 nm to about 800 nm, about 300 nm to about 700 nm, about 300 nm to about 600 nm, about 300 nm to about 500 nm, about 300 nm to about 400 nm, about 300 nm to about 390 nm, about 300 nm to about 380 nm, about 300 nm to about 370 nm, about 330 nm to about 380 nm, about 350 nm to about 380 nm, about 350 nm to about 370 nm, about 360 nm to about 380 nm, about 360 nm to about 370 nm, about 365 nm to about 375 nm, about 365 nm to about 370 nm, about 365 nm, or about 370 nm.

In certain embodiments, the concentration of the contrast agent is less than 0.9 micromolar, less than 0.8 micromolar, less than 0.7 micromolar, less than 0.6 micromolar, less than 0.5 micromolar, less than 0.4 micromolar, less than 0.3 micromolar, less than 0.2 micromolar, or less than 0.1 micromolar.

In certain embodiments, the concentration of the analyte is less than 1 micromolar, 0.9 micromolar, less than 0.8 micromolar, less than 0.7 micromolar, less than 0.6 micromolar, less than 0.5 micromolar, less than 0.4 micromolar, less than 0.3 micromolar, less than 0.2 micromolar, less than 0.1 micromolar, less than 0.01 micromolar, or less than 0.001 micromolar.

In certain embodiments, the signal induced by the contrast agent in the presence of the analyte or electromagnetic radiation that is at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, or at least 100% different from an MRI signal induced by the contrast agent under essentially identical conditions but in the absence of the analyte or electromagnetic radiation.

In certain embodiments, the method further comprises administering an MRI imaging agent or paramagnetic contrast agent of the present disclosure to a subject.

### Dosing Forms

In certain embodiments, the MRI imaging agent or paramagnetic contrast agent is a solid. In certain embodiments, the MRI imaging agent or paramagnetic contrast agent is a powder. In certain embodiments, the MRI imaging agent or paramagnetic contrast agent is a mixture of solid and liquid. In certain embodiments, the MRI imaging agent or paramagnetic contrast agent forms an emulsion when formulated for administration. In certain embodiments, the MRI imaging agent or paramagnetic contrast agent can be dissolved in a liquid to make a solution. In certain embodiments, the MRI imaging agent or paramagnetic contrast agent is dissolved in water to make an aqueous solution. In certain embodiments, the MRI imaging agent or paramagnetic contrast agent is a liquid for parental injection. In certain embodiments, the MRI imaging agent or paramagnetic contrast agent is a liquid for oral administration (e.g., ingestion). In certain embodiments, the MRI imaging agent or paramagnetic contrast agent is a liquid (e.g., aqueous solution) for intravenous injection. In certain embodiments, the composition is an MRI imaging agent or paramagnetic contrast agent (e.g., aqueous solution) for subcutaneous injection.

After appropriate formulation in a desired dosage, the MRI imaging agent or paramagnetic contrast agent of this disclosure can be administered to humans and other animals orally, parenterally, intracisternally, intraperitoneally, topically, bucally, or the like.

Compositions comprising the MRI imaging agents and paramagnetic contrast agents described herein can be prepared by any method known in the art of pharmacology. In general, such preparatory methods include the steps of bringing the agent into association with a carrier and/or one or more other accessory ingredients, and then, if necessary and/or desirable, shaping and/or packaging the product into a desired single- or multi-dose unit.

The compositions can be prepared, packaged, and/or sold in bulk, as a single unit dose, and/or as a plurality of single unit doses. As used herein, a “unit dose” is a discrete amount of the composition comprising a predetermined amount of the MRI imaging agent or paramagnetic contrast agent.

Relative amounts of the agent, an excipient, and/or any additional ingredients in a composition of the present disclosure will vary, depending upon the identity, size, and/or condition of the subject treated and further depending upon the route by which the composition is to be administered. By way of example, the composition may comprise between 0.1% and 100% (w/w) the agent.

Acceptable excipients used in the manufacture of provided compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the present disclosure are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this disclosure.

Also encompassed by the disclosure are kits. The kits provided may comprise a composition or agent described herein and a container (e.g., a vial, ampule, bottle, syringe, and/or dispenser package, or other suitable container). In some embodiments, provided kits may optionally further include a second container comprising an excipient for dilution or suspension of a composition or agent described herein. In some embodiments, the agent described herein provided in the first container and the second container are combined to form one unit dosage form.

Thus, in one aspect, provided are kits including a first container comprising an agent or composition described herein. In certain embodiments, the kits are useful for obtaining a magnetic resonance image of a subject.

In certain embodiments, a kit described herein further includes instructions for using the kit. A kit described herein may also include information as required by a regulatory agency such as the U.S. Food and Drug Administration (FDA).

### Commercial Applications

In certain embodiments, the agents and methods described herein are useful for both basic biological discovery and drug development. In certain embodiments, employment of the agents and methods described herein provide functional physiological understanding of the biology of major disease areas, and lead to the discovery of addressable functional mechanisms in health and disease. For example, pharmacological screening and characterization will benefit from the advantages of the agents and methods described herein. Using MRI as a readout will facilitate the study of drug effects in the same way as the study of natural physiological phenomena.

In other embodiments, such as clinical diagnostic imaging, the agents and methods described herein may be applied to molecular imaging, e.g., to visualize molecular concentrations or activities, for example of neurotransmitters, blood glucose, tumor-associated analytes, or physiologically relevant ions.

### Definitions

The term “analyte-recognition entity” refers to a molecule or assembly of molecules defined by a chemical structure or assembly of chemical structures respectively in which the entity has the ability to recognize an analyte of interest. In certain embodiments, the entity recognizes the analyte directly, such as by formation of a chemical bond (e.g., hydrogen bond, covalent bond, ionic bond). In certain embodiments, the entity recognizes the analyte indirectly. In certain embodiments, the analyte-recognition entity is an electromagnetic radiation-affected entity, as defined herein.

The term “electromagnetic radiation” refers to waves of the electromagnetic (EM) field, propagating through space, carrying electromagnetic radiant energy. Electromagetic radiations includes radio waves, microwaves, infrared, (visible) light, ultraviolet, X-rays, and gamma rays. All of these waves form part of the electromagnetic spectrum.

The term “electromagnetic radiation-affected entity” refers to a molecule or assembly of molecules defined by a chemical structure or assembly of chemical structures respectively in which the entity undergoes a transformation upon exposure to electromagnetic radiation. In certain embodiments, the entity comprises a specific chemical group or moiety that undergoes a chemical transformation upon exposure to electromagnetic radiation. In certain embodiments, the specific chemical group or moiety isomerizes upon exposure to electromagnetic radiation.

The term “liposome” refers to a spherical vesicle having an aqueous solution core surrounded by a hydrophobic membrane, in the form of a lipid bilayer. Hydrophilic solutes dissolved in the core cannot readily pass through the bilayer. Liposomes are most often composed of phospholipids, but may also include other lipids, such as egg phosphatidylethanolamine, so long as they are compatible with lipid bilayer structure. Liposomes can be prepared by disrupting biological membranes (e.g., by sonication).

The term “molecular entity” refers to a molecule or assembly of molecules defined by a chemical structure or assembly of chemical structures respectively. A molecular entity may be a single molecule or a complex of plural molecules, and may include one or more targeting moiety. In certain embodiments, the molecular entity is a polyethylene glycol chain, e.g., to facilitate movement of the liposome to or near physiological locations of interest.

The term “targeting moiety” refers to a member of a specific binding pair, i.e., a member of a pair of molecules, wherein one of the pair of molecules has an area on its surface, or a cavity that specifically binds to, and is, therefore, defined as complementary with a particular spatial and polar organization of the other molecule, so that the pair have the property of binding specifically to each other. Examples of types of specific binding pairs are antigen-antibody, biotin-avidin, hormone-hormone receptor, receptor-ligand, enzyme-substrate, and IgG-protein A. In certain embodiments, targeting moieties may be a protein, a small molecule, a protein, a peptide, a nucleotide, a polynucleotide, a nucleic acid, or an antibody.

The term “small molecule” refers to molecules, whether naturally-occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “complex” or “coordination complex” refers to an association of at least one atom or ion (which is referred to as a “central atom,” “central ion,” or “acceptor,” and is usually a metallic cation) and a surrounding array of bound ligands or donors). A complex may include one or more donors, which can be the same or different. A complex may also include one or more acceptors, which can be the same or different.

The term “ligand” refers to an ion or molecule that binds to a central atom or central ion (e.g., a central metal atom or central metal ion) to form a coordination complex (e.g., a coordinate covalent bond (e.g., ligands may donate electrons from a lone electron pair into an empty orbital of the central atom or central ion))) and are referred to as being “coordinated” to the central atom or central ion. There are also organic ligands such as alkenes whose n-bonds can coordinate to empty orbitals of an acceptor. Ligands are usually electron donors, and the central atom or ion is electron acceptors. The bonding between the central atom or ion and the ligand typically involves formal donation of one or more of the ligand's electron pairs. The nature of such bonding can range from covalent to ionic, and the bond order can range from one to three. One central atom or ion may bind to one or more ligands of the same or different type. A ligand may be capable of binding a central atom or ion through multiple sites, usually because the ligand includes lone pairs on more than one atom of the ligand. Ligands in a complex may affect the reactivity (e.g., ligand substitution rates and redox) of the central atom or ion. Exemplary ligands include charge-neutral ligands (“ligand molecules,” e.g., CH₃CN, amides (e.g., N,N-dimethylformamide (DMF), N,N-dimethylacetamide (DMA), or N-methyl-2-pyrrolidone (NMP)), dimethyl sulfoxide (DMSO), amines (e.g., ammonia; ethylenediamine (en); pyridine (py); 2,2′-bipyridine (bipy); and 1,10-phenanthroline (phen)), phosphines (e.g., PPh₃), ethers (e.g., tetrahydrofuran (THF), 2-methyl-tetrahydrofuran, tetrahydropyran, dioxane, diethyl ether, methyl t-butyl ether (MTBE), dimethoxyethane (DME), and diglyme), ketones (e.g., acetone and butanone), chlorohydrocarbons (e.g., dichloromethane (DCM), chloroform, carbon tetrachloride, and 1,2-dichloroethane (DCE)), esters (e.g., propylene carbonate and ethyl acetate), CO, N₂, water, and alkenes) and anionic ligands (“ligand ions,” e.g., halides, hydride, alkyls, S₂⁻, S—CN⁻, O—NO₂⁻, N—N₂⁻, O—H⁻, [O—C(═O)—C(═O)—O]₂⁻, O—N—O⁻, N═C═S⁻, CN⁻).

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “endogenous” refers to a substance that originates within the body of a living organism.

The term “subject” refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “tissue” refers to any biological tissue of a subject (including a group of cells, a body part, or an organ) or a part thereof, including blood and/or lymph vessels, which is the object to which a compound, particle, and/or composition of the present disclosure is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

### EXAMPLES

Abbreviations: 1,2-Dipalmitoyl-sn-Glycero-3-Phosphocholine (DPPC); 1,2-Dipalmitoyl-sn-Glycero-3-Phosphoglycerol (DPPG); Polyethylene Glycol-Modified 1,2-Distearoyl-sn-Glycero-3-Phosphoethanolamine (DSPE-PEG); 1-palmitoyl-2-oleoyl-sn-glycero-3-phosphocholine (POPC); Liposomal Nanoparticle Reporter (LisNR); Longitudinal Relaxation Rate (R1); Longitudinal Relaxation Time (T1); Longitudinal Relaxivity (r1); Magnetic Resonance Imaging (MRI); Ultraviolet (UV)

### Construction of Photoresponsive Liposomal MRI Probes

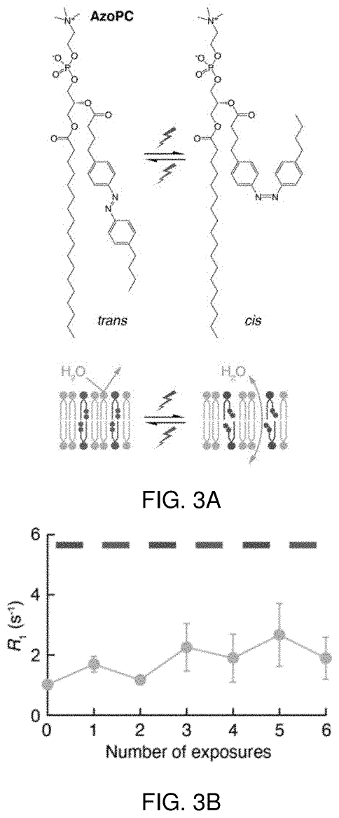

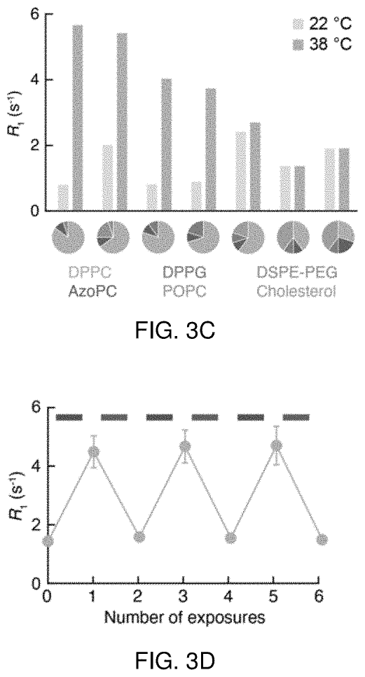

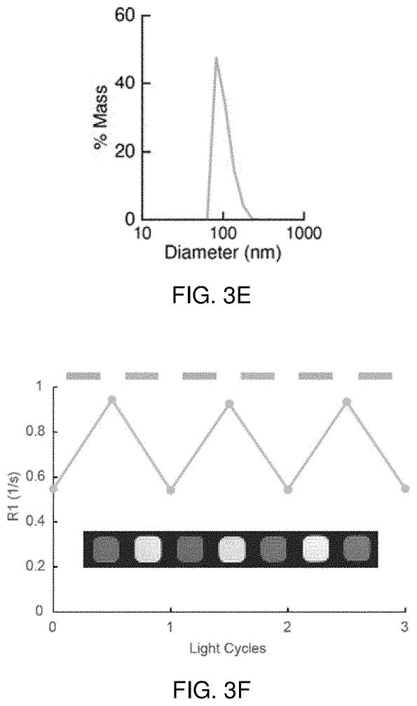

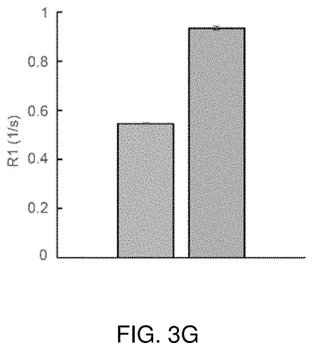

To produce light-responsive liposomal nanosensors (Light-LisNRs), 1-stearoyl-2-(4-(n-butyl)phenylazo-4′-phenylbutyroyl)phosphocholine (AzoPC), a synthetic phosphatidylcholine variant that contains the well-characterized photoisomerizable moiety azobenzene in one of its fatty acyl chains. was utilized. Absorption of blue light (λₘₐₓ=460 nm) by AzoPC favors an extended trans conformation that emulates that of naturally occurring saturated lipids, whereas absorption of ultraviolet light (UV, λₘₐₓ=370 nm) switches AzoPC into a kinked cis conformation that disrupts monolayer structure, promoting transmembrane exchange (FIGS. 3B-C). Liposomes using 25% AzoPC, 70% of the saturated lipid 1,2-dipalmitoyl-sn-glycero-3-phosphocholine (DPPC), and 5% 1,2-distearoyl-sn-glycero-3-phosphoethanolamine modified with polyethylene glycol (DSPE-PEG) were formulated. These components were dissolved into a 220 mM solution of the neutral gadolinium-containing contrast agent gadoteridol, extruded through a 100 nm-mesh filter, and desalted to remove unencapsulated agent (FIG. 3C). The resulting candidate Light-LisNRs were evaluated by dynamic light scattering, which revealed a mean hydrodynamic diameter of 92 t 3.6 nm.

Photosensitivity of initial Light-LisNRs was investigated by measuring their responses to blue and UV illumination using MRI at 7 T and room temperature in microtiter plates. Relaxation rates were recorded between serially alternating 1 or 3-minute epochs of 460 nm and 370 nm irradiation delivered with approximate intensities of 0.25 and 0.1 mW/mm², respectively. FIGS. 3D-E show that ˜30 nM probes, corresponding to 2.2 mM Gd³⁺, undergo clear light-dependent switching. Each UV epoch produced an increase in R₁, corresponding to T₁-weighted MRI signal brightening, and each blue illumination epoch resulted in decreases in R₁ and MRI signal, consistent with the higher liposome permeability expected in the presence of the UV-favored cis-AzoPC conformation. Over multiple cycles, however, a trend toward higher relaxation rates was evident. Gel filtration and elemental analysis with ICP-MS revealed that multiple switching cycles resulted in significant leakage of gadoteridol from the liposomes, explaining the instability in MRI results. More consistent light-dependent behavior could be produced by substantially reducing the AzoPC content of the LisNR membranes. Light-LisNRs formulated with 5% AzoPC and 90% DPPC underwent highly reproducible switching between low- and high-relaxation states. Although Light-LisNRs containing 5% AzoPC are more stable, however, they show a much lower mean change in R₁ of 0.088±0.002 s⁻¹ after each UV illumination step, compared with mean ΔR₁ values of 0.843±0.173 s⁻¹ for the 25% AzoPC-based probes.

To combine the high relaxivity changes obtained with LisNRs incorporating 25% AzoPC with the stability exhibited by liposomes containing 5% AzoPC, a set of liposome compositions for relaxation enhancement and stability to heating were screened (FIG. 3F). In addition to varying concentrations of AzoPC, DPPC, and DSPE-PEG, incorporation of 1,2-dipalmitoyl-sn-glycero-3-phosphoglycerol (DPPG), an uncharged alternative to DSPE-PEG, and cholesterol, which promotes integrity of curved bilayers, were explored. Of the conditions surveyed, liposomes containing 20% AzoPC, 30% DPPC, 10% DPPG, and 40% cholesterol displayed a superior combination of low blue-state relaxivity and stability, and further experiments with liposomes of this composition were conducted.

### Characterization of Light-LisNR Response Properties

The Light-LisNRs displayed markedly enhanced light switching behavior in MRI. Alternating blocks of blue and UV light produced consistent R₁ values of 1.52 t 0.040 s⁻¹ and 4.52±0.096 s⁻¹, respectively, with negligible contrast agent leakage. Given the effective gadolinium concentration of 2.2 mM, these results correspond to reproducible r₁ values of 0.69 mM⁻¹s⁻¹ and 2.05 mM⁻¹s⁻¹ for the blue and UV states of the sensors, expressed with respect to gadolinium concentration at room-temperature and 7 T. This robust light-dependent relaxivity difference corresponds to mean water residence times of 7.73 ms and 1.23 ms in the blue and UV states.

Per metal ion, the Δr₁ value of 1.36 mM⁻¹ s⁻¹ documented for Light-LisNRs compares favorably to light-switchable contrast agents that use a Ni²⁺ spin switching mechanism (Δr₁=0.126 mM⁻¹s⁻¹, 7 T, room temperature, in DMSO) or Gd³⁺ inner sphere modulation mechanism (Δr₁=0.3 mM⁻¹s⁻¹).

### Validation of Light-LisNRs by In Vivo Imaging

These results indicate that Light-LisNRs are appropriate for light mapping measurements in live animals, a biologically important capability that previous photosensitive MRI agents have not achieved. How photons spread in tissue determines which structures are probed by popular experimental techniques that involve light delivery and detection via optical fibers and other means. Light mapping could be critical for experimental interpretation in such paradigms, but the relevant volumetric profiles of light administration or reception are extremely difficult to determine in intact subjects. As a result, previous estimates of light spreading in tissue have been based on theoretical models, ex vivo tests, or point measurements that do not provide direct three-dimensional information.

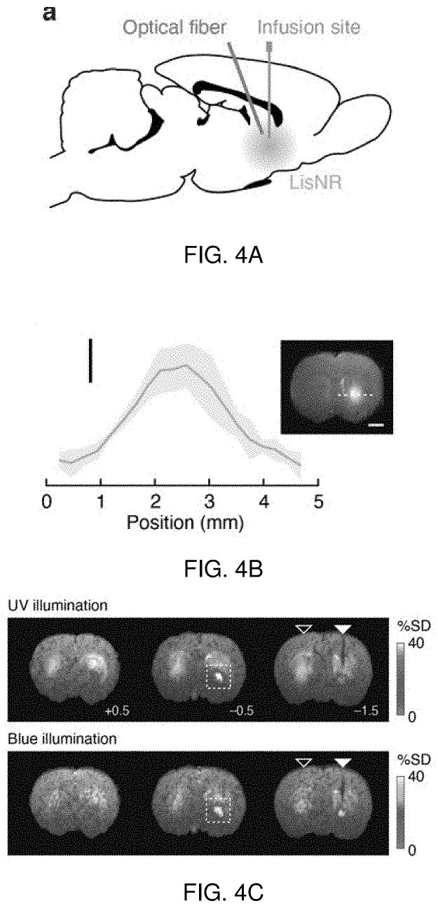

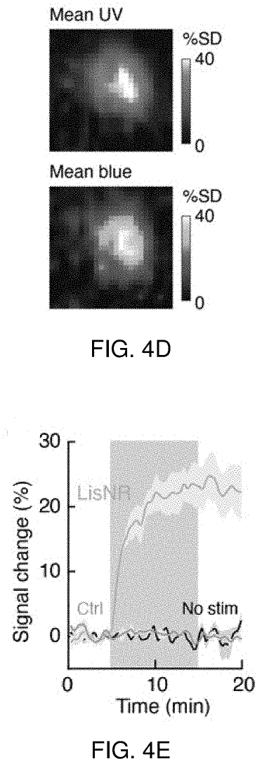

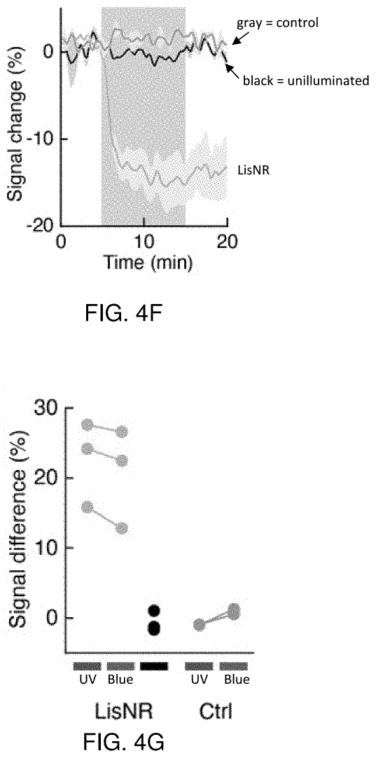

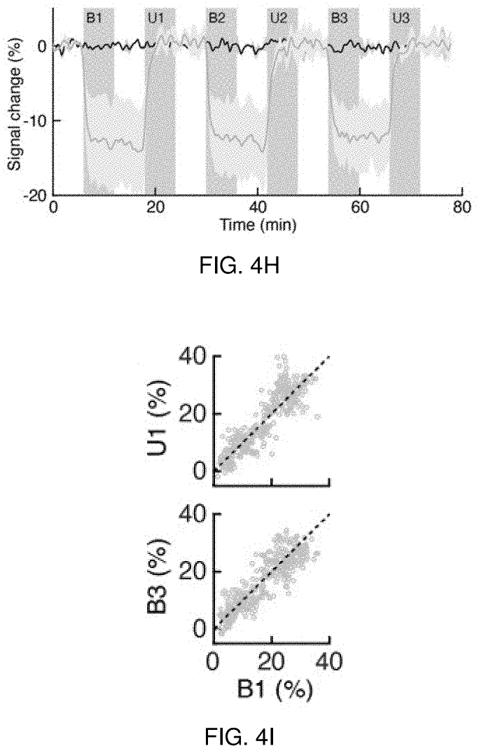

To assess the performance of the Light-LisNR in vivo, the probe was injected into live rat brain, targeting striatal tissue in the neighborhood of an implanted optical fiber similar to devices used widely in optogenetics and photometry experiments (FIG. 4A). Using T₁-weighted gradient echo MRI at 9.4 T, it was observed that LisNRs injection results in signal increases of over 5% (FIG. 4B). This indicates that Light-LisNRs spread effectively in tissue despite their ˜100 nm diameters, perhaps because of their ability to deform during convective infiltration of the parenchyma during injection. Probe contrast distributions were stable over time following delivery.

Functionality of Light-LisNRs in tissue was demonstrated by illuminating injected brain regions with blue or UV light delivered through the implanted fibers during acquisition of serial spin echo MRI scans. Delivery of blue light at first produced no significant effects, indicating that the infused LisNRs were indeed in their low relaxivity state with AzoPC molecules in the trans conformation. Application of UV light, however, produced clear MRI signal increases in the LisNR-injected regions of the animals (FIGS. 4C-4D). The time courses of UV responses in these areas are shown in FIG. 4E. Subsequent delivery of blue light reversed these signal changes with a mean time constant and an amplitude that were statistically identical to the UV-induced effect (FIGS. 4F-4H). MRI signal changes elicited by shorter blocks of stimulation closely track time courses observed during longer illumination periods, indicating that temporal characteristics of Light-LisNR responses in vivo are limited by photon delivery rates, as opposed to hysteretic behavior of the probes themselves. Forward and reverse contrast changes could be produced repeatedly, using alternating blocks of UV and blue light, with a coefficient of variation that indicates stable performance (FIG. 4I). The spatial profiles of UV and blue light-induced signal changes are highly reproducible within individual animals. Control experiments omitting illumination or using light-insensitive liposomes did not reveal significant MRI signal changes (FIGS. 4E, 4H). Both temporal behavior of the probes and the exhibited dynamic range were also consistent results obtained in vitro, indicating that Light-LisNR function was not compromised following injection into the rat brain.

### Volumetric Mapping of Light Distribution in Tissue

The stable performance of Light-LisNRs in rat brain indicates their suitability for quantitative measurements of light intensity distribution in tissue. To form light maps, in vitro measurements of photoresponse rate vs. light dose as a basis for quantifying local illumination levels in tissue were used. This approach exploits the monoexponential dependence of LisNR relaxivity on light intensity and results in estimates independent of the probe concentration in each voxel. Voxel-level MRI signal change time courses in probe-injected regions were first converted to estimated R₁ changes as a function of time. These R₁ time courses were then fit to exponential decay functions to determine light-dependent time constants τ_{obs} for each voxel; voxels with poor fit quality were eliminated from the analysis. For surviving voxels, the local light intensity was estimated as 10 mW/mm²×τ₁₀/τ_{obs} where τ₁₀ is the time constant for responses to 10 mW/mm² incident light measured in vitro.

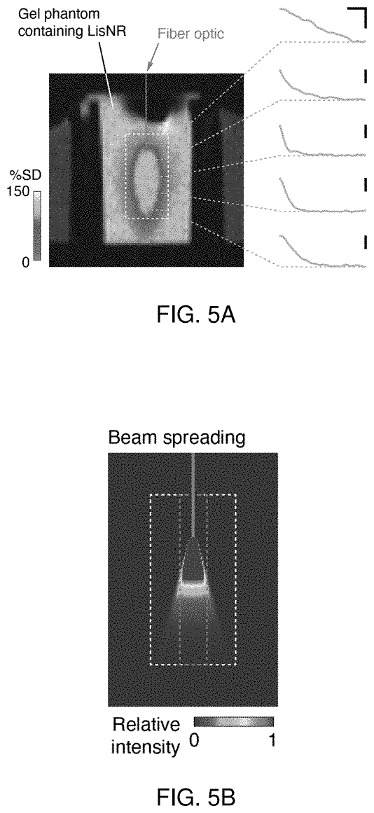

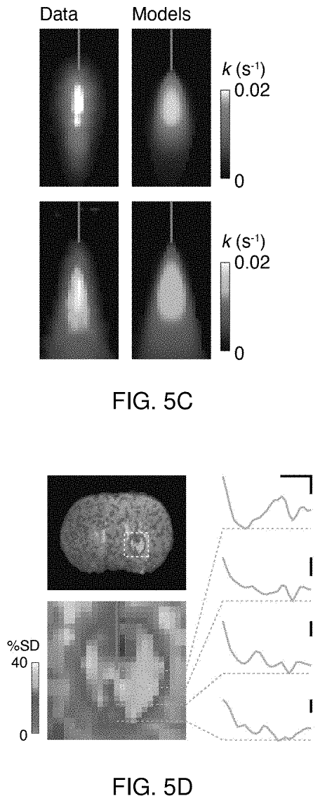

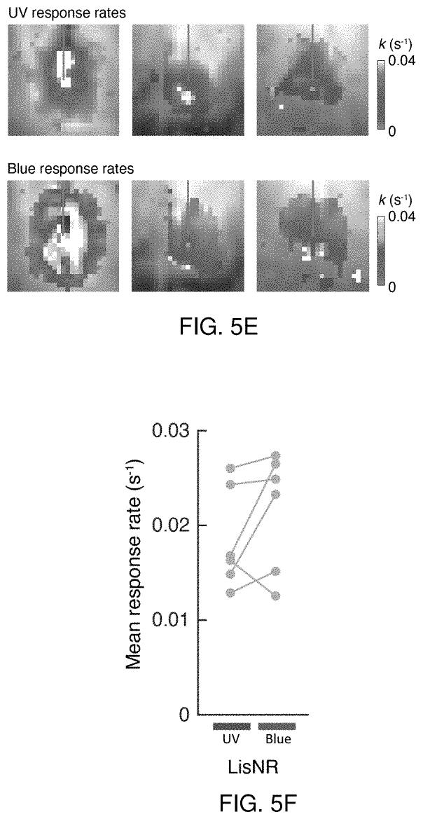

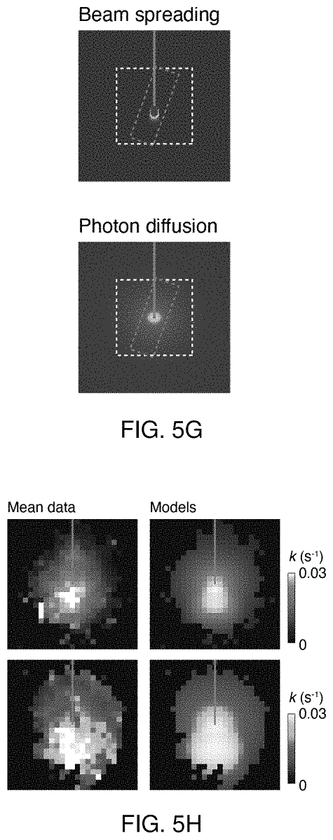

Processing steps involved in quantitative light mapping are illustrated for a representative dataset in FIG. 5A. Normalized illumination intensity distributions obtained using the procedure are shown in the average light maps depicted in FIG. 5B. The mean intensity as a function of distance from the tip. parallel to the fiber direction, is plotted in FIG. 5C, and indicates an attenuation length constant that is slightly longer for blue light than UV light. Lateral spreading of the applied light can be gauged from the radial intensity profile from near the fiber tip (FIG. 5D). These results are consistent with the greater tissue penetration and lesser scattering expected for blue light, compared with UV.

To estimate attenuation and scattering parameters using the light mapping results, these data were fit to an analytical beam spreading model previously used to approximate optogenetic stimulation profiles (FIG. 5E). The model approximated the data well. In vivo light mapping data deviated from ideality in terms of its spatial unevenness and variability among animals, as quantified by the average pairwise root mean squared difference between pairs of maps under blue or UV illumination conditions. Equivalent data measured in a homogeneous gel phantom model (FIG. 5F) showed significantly lower variability, and also fit the beam spreading model better. This indicates that tissue microstructural characteristics substantially distort the propagation of light with respect to theoretical behavior in isotropic media.

### Conclusion

The above examples introduce a sensor architecture for MRI in which reversible analyte-dependent modulation of liposome membrane permeability regulates contrast effects arising from encapsulated contrast agent molecules. This mechanism is demonstrated in the context of photosensitive Light-LisNR probes, which harness the large amplification factor afforded by the LisNR contrast principle to produce robust and quantifiable light-dependent responses in vitro and in vivo. Light-LisNRs produced volumetric maps of light propagation through complex mammalian brain tissue, revealing phenomenology of broad relevance to optical experimentation in living subjects.

### Methods

### Animal Procedures

All animal procedures were conducted in accordance with National Institutes of Health guide-lines and with the approval of the MIT Committee on Animal Care. Experiments were performed on 9 male Sprague Dawley rats weighing between 350 and 450 g.

### Modeling

To model the relationship between R1, MRI signal, and bilayer water permeability for paramagnetic liposomes, we used a two compartment model. This model is valid for relatively small volume fractions (f≤10%) as it neglects the signal contribution of intra-liposomal water protons. This assumption is valid for all experimental conditions tested as liposomes were used at f˜0.1-1%. This model also assumes that water exchange is not diffusion-limited, a valid assumption in all cases as the time it takes for a water molecule to diffuse a characteristic distance of 100 nm is approximately 4 microseconds. The following equations, derived from the model, relate R1 values to the average lifetime of water molecules within liposomes (T) and the lipid bilayer water permeability (P). Related variables include liposome diameter (d), volume (V), surface area (S), and volume fraction (f); background R1 (R1b), the paramagnetic metal complex contribution to R1 (R1para), the relaxivity (r1para) and intraliposomal concentration (Cpara) of the paramagnetic metal complex, and the intraliposomal T1 (T1ves).

P=V/Sτ=d/6τ (2.1)

T1ves=1/(r1para*Cpara)+1/R1b (2.2)

R1para≡R1−R1b=f/(T1ves+(d/6*P)) (2.3)

To estimate MRI signal changes, we used the signal equation for spin echo pulse sequences, assuming the echo time is sufficiently short to make T2-weighted signal contributions negligible.

Signal ∝(1 −e^{-R1*TR}) (2.4)

### Light Sensitivity Required to Sense Luciferase Activity

We focus on Gaussia luciferases (GLuc), which are particularly bright and are spectrally-compatible with azobenzene photoisomerization (peak emission ˜480 nm). Light output from existing GLuc variants is reported to be 7-30 photons/s/molecule. We approximate the illumination intensity by considering a cubic volume element and estimating the total photon output as the photon flux through one face of the cube (⅙ of the total light output). In a 1 μL volume (face area 0.01 cm²), assuming an effective luciferase concentration of 100 nM, GLuc could generate ˜3e13 photons/s/cm².

### Liposome Synthesis and Purification

Unilamellar paramagnetic liposomes (LUVETs) were made using the thin film rehydration and extrusion method and were purified by size exclusion column chromatography. Liposome concentrations were determined by ICP-MS.

Unless otherwise stated, lipids were purchased from Avanti Polar Lipids (Alabaster, Ala.). AzoPC was kindly synthesized and provided by Johannes Morstein and Dirk Trauner (NYU). Lipids (1.65e-6 mol total) were co-dissolved in chloroform, with the exception of phosphotidyl glycerol which was dissolved in 97/2/1 (v/v) chloroform/methanol/water, and dried overnight under high vacuum. For long term storage, the resulting lipid films were kept at −20° C. in sealed vials (PTFE septum with parafilm) in a sealed secondary container with calcium sulfate. ProHance (Gadoteridol) was purchased from MIT Pharmacies and diluted to 220 mM gadolinium with distilled water. Gadoteridol solution (1.1 ml, 220 mM gadolinium) was added to a lipid film aliquot and the resulting solution was incubated at 58° C. using a water bath for at least 2 hours with brief vortexing every ˜10-20 minutes. The solution was then subjected to three freeze-thaw cycles with liquid nitrogen. To extrude liposomes, we used a liposome extrusion kit from Avanti Polar Lipids (Alabaster, Ala.) which uses 1 ml syringes to force the aqueous lipid solution through polycarbonate filters of defined pore size (≤200 nm for predominantly unilamellar liposomes). The lipid solution was forced through double-stacked filters 21 times while maintaining temperatures above the highest phase transition temperature of the individual lipid constituents (typically 60° C.) a heating block. The resulting liposome solution was purified by gravity flow size exclusion column chromatography using resin Sepharose CL-4B purchased from Cytiva (Marlborough, Mass.) and buffer (10 mM HEPES (pH 7.4 with HCl), 139 mM NaCl) to remove unencapsulated gadoteridol. The resulting purified liposome solutions were quantified using an Agilent ICP-MS instrument (MIT CEHS Core Facility) using standards from 0-1000 ppb gadolinium with 10 ppb erbium as an internal standard. Liposome size were characterized by dynamic light scattering. The fraction of free contrast agent remaining after purification (or after heating for heat-stability tests), typically ˜3-5%, was determined by further size exclusion chromatography and ICP-MS of liposome-associated (early) and free contrast agent (late) fractions. Liposome solutions were stored in the dark at 4° C. (never frozen) for up to several months.

### Light Sources

Light was delivered in vitro using UV (365 nm, ˜8 mW/cm²) and blue (460 nm, ˜24 mW/cm²) LED flashlights (Amazon). Light intensity was determined using a digital optical power meter, UV-compatible sensor, and neutral density filters to decrease intensity, all purchased from Thorlabs (Newton, N.H.).

Light for in vivo experiments was delivered by a multimode optic fiber with a diameter of 200 μm and a 0.48 numerical aperture from Thorlabs (Newton, N.J.). After removing the cladding from the tip, the fiber delivering either ultraviolet or blue light for liposome activation/inactivation was inserted via a craniotomy and glued to the skull with biocompatible UV glue. The amount of glue for holding the fiber in place was kept at a necessary minimum for the fMRI experiments to reduce MR image distortions. During the MRI experiment the fiber was then connected to the respective light source, either a UV, 365 nm, LED from Thorlabs (Newton, N.J.) or a blue, 470 nm, LED from NPI (Tamm, Germany) via a SMA connector.

### In Vitro Imaging

MRI was performed on a 9.4 T BioSpec small animal scanner (Bruker, Billerica, Mass.) using a 70 cm inner diameter linear volume coil (Bruker). Scanner operation was controlled using the ParaVision 5.1 software (Bruker). T1-mapping experiments were performed using a series of MSME spin echo scans with echo time (TE)=11 ms, matrix size=256 by 256, field of view (FOV)=5 cm by 5 cm, slice thickness=2 mm and excitation angle=90° with repetitions times (TR) from 30 ms to 5 seconds. The number of scan averages was set such that total scan time at each TR was at least 7 minutes. Raw FID data was reconstructed and analyzed using a custom Matlab Mathworks (Natick, Mass.) script.

### In Vivo Imaging

Adult male Sprague-Dawley rats (350-450 g) were purchased from Charles River Laboratories (Wilmington, Mass.). After arrival, animals were housed and maintained on a 12 hour light/dark cycle and permitted ad libitum access to food and water. All procedures were carried out in strict compliance with National Institutes of Health guidelines, under oversight of the Committee on Animal Care at the Massachusetts Institute of Technology. Animals were anesthetized with isoflurane (3% for induction, 2% for maintenance) and placed on a water heating pad from Braintree Scientific (Braintree, Mass.) to keep body temperature at 37° C. Animals were then fixed in a stereotaxic frame, and topical lidocaine was applied before a 3 cm lateral incision extending from bregma to lambda was made, exposing the skull. Craniotomies (0.5 mm) were drilled bilaterally over the caudate Putamen (CPu), 0.5 mm posterior and 3.0 mm lateral to bregma. 28-gauge infusion cannulae were lowered to 6.5 mm below the surface of the skull through each craniotomy and were held in place by the stereotactic arms. 15 μL of liposomes (f=2%), light-responsive (20% azoPC, 30% distearoylphosphatidylcholine, 40% cholesterol, 20% dipalmitoylphosphatidylglycerol) or non-responsive (20% 1-palmitoyl-2-oleoyl-sn-glycero-3-phosphocholine, 30% disteroylphosphatidylcholine, 40% cholesterol, 20% dipalmitoylphosphatidylglycerol) for control experiments, were infused over the course of 2 hours. Ten minutes after the injection, infusion cannulae were slowly removed from the brain, and the craniotomies were closed with bone wax (CP Medical, Inc. Portland, Oreg.). The fiber was lowered through a third craniotomy (3.0 mm posterior and +3.00 mm lateral to bregma), with an angle of 21° in anterior-posterior direction until 7.5 mm below the surface of the skull, to reach the location of where liposomes were previously infused. Animals were then transferred into a custom rat imaging cradle, fixed with ear bars and bite bar, maintained under 2% isoflurane anesthesia, and kept warm using a recirculating water heating pad for the duration of imaging.

MRI was performed on a Bruker 9.4 T BioSpec small animal scanner (Billerica, Mass.) using a transmit-only 70 cm inner diameter linear volume coil made by Bruker (Billerica, Mass.) and a receive-only 3×1 array-coil with openings through which the fiber was led through to then be connected to the respective light sources outside of the scanner room. Scanner operation was controlled using the ParaVision 6.01 software from Bruker (Billerica, Mass.).

T₁-mapping was carried out using a series of MSME spin echo scans with echo time (TE)=5 ms, matrix size=128 by 64, field of view (FOV)=30 by 18 mm, slice thickness=1 mm, with repetition times (TR) from 200 ms to 3 seconds. The number of scan averages was set such that total scan time at each TR was at least 2 minutes. Raw FID data was reconstructed and analyzed using a custom Matlab Mathworks (Natick, Mass.) script.

Multislice T1-weighted fast low-angle-shot MRI images were acquired to evaluate the spread of liposomes in the CPu with TE=4 ms, TR=200 ms, field of view (FOV)=30×18 mm, in-plane resolution 200×200 sm, and four coronal slices with slice thickness=1 mm. Sagittal T2-weighted TurboRARE images with TE=30 ms, TR=1000 ms, Rare Factor 8, Matrix size 256×256, FOV=25.6×25.6 and in-plane resolution of 100×100 μm were used to evaluate the position of the fiber.

Rapid acquisition with refocused echoes (RARE) pulse sequences were used for functional scans with the following parameters: FOV=30×18 mm, slice thickness=1 mm, matrix size=128×64, and RARE factor=4 (with 5 ms echo spacing). Functional scans of different lengths with different ON and OFF periods of either blue or UV LED light were performed and their signal change and functional maps were assessed.

All quantitative data analyses were performed using MATLAB by Mathworks (Natick, Mass.). Statistical comparisons were performed using Student's t-test, unless otherwise specified.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “mol %” is an abbreviation of mole or molar percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims and clauses, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

For reasons of completeness, various aspects of the present disclosure are set out in the following clauses:

Clause A. An MRI imaging agent, comprising: a liposome encapsulating an MRI contrast agent; and an analyte-recognition entity associated with the liposome that affects water permeability of the liposome in response to proximity of an analyte.

Clause B. A paramagnetic contrast agent comprising: a liposome; and an analyte-recognition entity associated with the liposome that affects water permeability of the liposome in response to proximity of an analyte.

Clause C. An MRI imaging agent or paramagnetic contrast agent as in any preceding clause, wherein the liposome comprises an entity affected by electromagnetic radiation to switch between a first conformation providing a first level of water permeability across the lipid bilayer of the liposome, and a second conformation providing a second level of water permeability across the lipid bilayer.

Clause Z. An MRI imaging agent or paramagnetic contrast agent as in any preceding clause, wherein the liposome comprises a water-permeable molecular channel that contains or binds the analyte recognition entity.

Clause Y. An MRI imaging agent or paramagnetic contrast agent as in clause Z, wherein the water-permeable molecular channel is a polypeptide.

Clause D. An MRI imaging agent or paramagnetic contrast agent as in any preceding clause, wherein the liposome comprises a tethered moiety that can bind the analyte recognition entity.

Clause E. An MRI imaging agent or paramagnetic contrast agent as in clause D, wherein the tethered moiety is a neurotransmitter or neurotransmitter analog.

Clause F. A method comprising in a medium, exposing an analyte to an MRI contrast agent comprising a recognition entity for the analyte, wherein the concentration of the MRI contrast agent is less than 1 micromolar; and recording an MRI signal influenced by the contrast agent in the presence of the analyte that is at least 1% different from an MRI signal influenced by the contrast agent under essentially identical conditions but in the absence of the analyte.

Clause G. An MRI imaging agent or paramagnetic contrast agent, or method, as in any preceding clause, wherein the analyte-recognition entity reversibly affects water permeability of the liposome in response to proximity of an analyte.

1. An MRI imaging agent, comprising:
a liposome encapsulating an MRI contrast agent; and
an analyte-recognition entity associated with the liposome that reversibly affects water permeability of the liposome in response to proximity of an analyte, or an electromagnetic radiation-affected entity associated with the liposome that reversibly affects water permeability of the liposome in response to electromagnetic radiation.

2. A paramagnetic contrast agent comprising:
a liposome; and
an analyte-recognition entity associated with the liposome that reversibly affects water permeability of the liposome in response to proximity of an analyte, or an electromagnetic radiation-affected entity associated with the liposome that reversibly affects water permeability of the liposome in response to electromagnetic radiation.

3-4. (canceled)

5. The MRI imaging agent of claim 1, wherein the liposome comprises a water-permeable molecular channel spanning a lipid bilayer of the liposome.

6. The MRI imaging agent of claim 5, wherein the water-permeable molecular channel contains or binds the analyte recognition entity.

7. (canceled)

8. The MRI imaging agent of claim 5, wherein the water-permeable molecular channel is or comprises a transmembrane protein, a polypeptide, a phospholipid, a fatty acid chain, or a synthetic organic compound.

9-10. (canceled)

11. The MRI imaging agent of claim 1, wherein the liposome comprises a phospholipid, cholesterol, or a combination thereof.

12. The MRI imaging agent of claim 1, wherein the liposome comprises a phosphatidylglycerol, a phosphatidylethanolamine, a phosphatidylcholine, a phosphatidylserine, or cholesterol, or a combination thereof.

13-21. (canceled)

22. The MRI imaging agent of claim 1, wherein the contrast agent comprises a metal.

23-27. (canceled)

28. The MRI imaging agent of claim 6, wherein the liposome comprises a tethered moiety that binds the analyte recognition entity.

29. The MRI imaging agent of claim 28, wherein the tethered moiety is conjugated to the water-permeable molecular channel.

30-32. (canceled)

33. The MRI imaging agent of claim 1, wherein the analyte-recognition entity is bound to the surface of the liposome.

34. The MRI imaging agent of claim 5, wherein the water-permeable molecular channel contains the electromagnetic radiation-affected entity.

35. The MRI imaging agent of claim 1, wherein the electromagnetic radiation-affected entity is affected by electromagnetic radiation to switch between a first conformation providing a first level of water permeability across the lipid bilayer of the liposome, and a second conformation providing a second level of water permeability across the lipid bilayer.

36-37. (canceled)

38. The MRI imaging agent of claim 34, wherein the water-permeable molecular channel comprises a phospholipid having a modified fatty acid chain.

39. The MRI imaging agent of claim 38, wherein the modified fatty acid chain comprises a photoisomerizable moiety.

40. The MRI imaging agent of claim 39, wherein the modified fatty acid chain comprises an azobenzene moiety.

41. The MRI imaging agent of claim 40, wherein the water-permeable channel comprises 1-stearoyl-2-(4-(n-butyl)phenylazo-4′-phenylbutyroyl)phosphocholine (AzoPC).

42. The MRI imaging agent of claim 1, wherein the electromagnetic radiation is light.

43. (canceled)

44. A method of magnetic resonance imaging, the method comprising
in a medium, exposing an analyte or electromagnetic radiation to the MRI imaging agent of claim 1, wherein the concentration of the contrast agent is less than 1 micromolar; and
recording an MRI signal induced by the contrast agent in the presence of the analyte or electromagnetic radiation that is at least 1% different from an MRI signal induced by the contrast agent under essentially identical conditions but in the absence of the analyte or electromagnetic radiation.

45-47. (canceled)

48. A kit comprising the MRI imaging agent of claim 1 and instructions for use.
